(19) United States
(12) Patent Application Publication
(10) Pub. No.: US 2004/0168214 A1
(43) Pub. Date: Aug. 26, 2004

(54) Process for increasing the yield in plants by expressing stably integrated recombinant polypeptides

(75) Inventors: Marion Kwart, Berlin (DE); Jorg Riesmeier, Berlin (DE); Lothar Willmitzer, Berlin (DE)

Correspondence Address: BIRCH STEWART KOLASCH & BIRCH, PO BOX 747, FALLS CHURCH, VA, 22040-0747, US

(73) Assignee: Max-Planck-Gesellschaft zur Forderung der Wissenschaften e.V.

(21) Appl. No.: 10/749,453

(22) Filed: Dec. 30, 2003

Related U.S. Application Data
(62) Division of application No. 09/485,187, filed on Jun. 26, 2000, now abandoned.
Application No. 09/485,187 is a 371 of international application No. PCT/EP98/04878, filed on Aug. 5, 1998.

(30) Foreign Application Priority Data
Aug. 7, 1997 (DE) 197 34 218.3

(51) Int. Cl. A01H 1/00; C12N 15/82; C07H 21/04

(52) U.S. Cl. 800/284; 800/294; 536/23.2

(57) ABSTRACT

A process for increasing plant yield is described. Recombinant constructs containing polypeptides are expressed under the control of companion cell specific promoters. This results in improved phloem loading and leads to increases in biomass.

This application is a Divisional of co-pending Application No. 09/485,187, filed on Jun. 26, 2000 the entire contents of which are hereby incorporated by reference and for which priority is claimed under 35 U.S.C. § 120. Application No. 09/485,187 is the national phase under U.S.C. § 371 of PCT International Application No. PCT/EP98/04878 which has the International filing date of Aug. 5, 1998, which designated the United States of America. This application claims priority of Application No. 197 34 218.3 filed in Germany on Aug. 7, 1997 under 35 U.S.C. § 119.

### DESCRIPTION

The present invention relates to processes for increasing the yield in plants, recombinant nucleic acid molecules used for these processes, their uses as well as to plants with an increased yield.

In the field of agriculture and forestry constantly efforts are being made to produce plants with an increased yield, in particular in order to guarantee the supply of the constantly increasing world population with food and to guarantee the supply of reproducible raw materials. Conventionally, it is tried to obtain plants with an increased yield by breeding, which is, however time-consuming and labor-intensive. Furthermore, appropriate breeding programs have to be performed for each relevant plant species.

Progress has partly been made by the genetic manipulation of plants, that is by introducing into and expressing recombinant nucleic acid molecules in plants. Such approaches have the advantage of usually not being limited to one plant species but being transferable to other plant species. In EP-A 0 511 979, e.g., it was described that the expression of a prokaryotic asparagine synthetase in plant cells inter alia leads to an increased biomass production.

In WO 96/21737, e.g., the production of plants with an increased yield by the expression of deregulated or unregulated fructose-1,6-bisphosphatase due to the increase of the photosynthesis rate is described.

Nevertheless, there still is a need of generally applicable processes for improving the yield in plants interesting for agriculture or forestry.

Therefore, the problem underlying the present invention is to provide further processes for increasing the yield in plants.

This problem is solved according to the invention by providing the embodiments characterized in the claims.

Therefore, the present invention relates to a process for increasing the yield in plants, characterized in that recombinant DNA molecules stably integrated into the genome of plants are expressed, comprising

(a) a region allowing the transcription specifically in the companion cells; and operatively linked thereto

(b) a nucleotide sequence encoding a polypeptide selected from the group consisting of:

(i) proteins with an enzymatic activity that cleaves sucrose;

(ii) sucrose transporters;

(iii) proteins the activity of which leads to the stimulation of the proton gradient located at the plasma membrane of plant cells; and

(iv) citrate synthases (E.C. 4.1.3.7).

It was surprisingly found that the expression of the above-described proteins specifically in the phloem of plants leads to a dramatic increase in yield.

The term “increase in yield” preferably relates to an increase of the biomass production, in particular when determined as the fresh weight of the plant. Such an increase in yield preferably refers to the so-called “sink” organs of the plant, which are the organs that take up the photoassimilates produced during photosynthesis. Particularly preferred are parts of plants which can be harvested, such as seeds, fruits, storage roots, roots, tubers, flowers, buds, shoots, stems or wood. The increase in yield according to the invention is at least 3% with regard to the biomass in comparison to non-transformed plants of the same genotype when cultivated under the same conditions, preferably at least 10% and particularly preferred at least 20%.

The above-described proteins have in common that when they are expressed in the phloem their biological activity leads to an increased charging of the phloem with photoassimilates. In the context of the present invention photoassimilates are understood to be sugars and/or amino acids.

According to the invention the nucleotide sequence mentioned in (b) can usually encode a plant protein or a bacterial protein or a protein originating from fungi or animal organisms.

In a preferred embodiment the nucleotide sequence encodes a sucrose synthase (E.C. 2.4.1.13), preferably a plant sucrose synthase, in particular from Solanum tuberosum, and particularly preferred the type expressed in the tubers of S. tuberosum. Such sequences are, for example, described in Salanoubat and Belliard (Gene 60 (1987), 47-56) and are available in the EMBL gene bank under accession number X67125.

In a further preferred embodiment the nucleotide sequence encodes a sucrose phosphorylase (E.C. 2.4.1.7). Sequences encoding sucrose phosphorylase are, for example, known from WO 96/24679.

In another preferred embodiment the nucleotide sequence encodes an invertase (E.C. 3.2.1.26), preferably an invertase from a microorganism, in particular from a fungus of the genus Saccharomyces, preferably from S. cerevisiae. Particularly preferred are sequences encoding a cytosolic invertase (Sonnewald et al., Plant J. 1 (1991), 95-106).

According to the invention a sucrose transporter is understood to be a transporter transporting sucrose in plant systems across a membrane. Such a transporter preferably is of plant origin (for example EMBL gene bank accession number GI:21319). Particularly preferred the sequence described in (b) encodes a sucrose transporter from spinach (Spinacia oleracea), in particular with the sequence of the clone SoSUT1, as, e.g., described in Riesmeier et al. (EMBO J. 11 (1992), 4705-4713).

In a further preferred embodiment the protein that stimulates the proton gradient located at the plasma membrane is a proton ATPase. In this case, the sequence described in (b) preferably encodes a protein from a microorganism, in particular a fungus of the genus Saccharomyces, preferably from S. cerevisiae.

In a particularly preferred embodiment the sequence encodes the proton ATPase PMA1 from S. cerevisiae (Serrano et al., Nature 319 (1986), 689-693; EMBL gene bank) or a version of this proton ATpase from S. cerevisiae which is truncated at the 3′ end, in particular the ATPase ΔPAM1 as described in Example 3 of the present invention.

Alternatively, the nucleotide sequence can also encode a proton ATPase from plants, preferably a proton ATPase from Solanum tuberosum. Particularly preferred are sequences encoding the proton ATPase PHA2 from potato (Harms et al, Plant Mol. Biol. 26 (1994), 979-988; EMBL gene bank X76535) or a version of this proton ATPase from potato which is truncated at the 3′ end, in particular the ATPase ΔPHA2 as described in Example 4 of the present invention.

According to the invention the citrate synthase can be any citrate synthase, for example those from bacteria, fungi, animals or plants. DNA sequences encoding citrate synthase are known, for example, from the following organisms: Bacillus subtilis (U05256 and U05257), E. coli (VO1501), R. prowazekii (M17149), P. aeruginosa (M29728), A. anitratum (M33037) (see.Schendel et al., Appl. Environ. Microbiol. 58 (1992), 335-345 and references cited therein), Haloferax volcanii (James et al., Biochem. Soc. Trans. 20 (1992), 12), Arabidopsis thaliana (Z17455) (Unger et al., (1989) Plant Mol. Biol. 13 (1989), 411-418), B. coagulans M74818), C. burnetti (M36338) (Heinzen et al., Gene 109 (1990), 63-69), M. smegmatis (X60513), T. acidophilum (X55282), T. thermophila (D90117), pig (M21197) (Bloxham et al., Proc. Natl. Acad. Sci. USA 78 (1981), 5381-5385), N. crassa (M84187) (Ferea et al., Mol. Gen. Genet. 242 (1994), 105-110), S. cerevisiae (Z11113, Z23259, M14686, M54982, X00782) (Suissa et al., EMBO J. 3 (1984), 1773-1781) and potato (EP 95 91 3066.7).

The numbers in brackets are the corresponding accession numbers in the GenEMBL data base.

The nucleotide sequences according to the invention can generally encode any appropriate proteins from any organism, in particular from plants, fungi, bacteria or animals. The sequences preferably encode proteins from plants or fungi. Preferably, the plants are higher plants, in particular starch or oil storing useful plants, for example potato or cereals such as rice, maize, wheat, barley, rye, triticale, oat, millet, etc., as well as spinach, tobacco, sugar beet, soya, cotton etc. The fungi preferably are of the genus Saccharomyces, Schizosaccharomyces, Aspergillus or Neurospora, in particular Saccharomyces cerevisiae, Schizosaccharomyces pombe, Aspergillus flavus, Aspergillus niger or Neurospora crassa.

In a preferred embodiment of the process according to the invention the region mentioned in (a), which guarantees a companion cell specific transcription, is the promoter of the roIC gene from Agrobacterium rhizogenes. This promoter is, for example, described in Schmülling et al. (Plant Cell (1989), 665-671) and Kühn (Characterization and localization of the sucrose carrier SUT1 in Solanaceae, Doctoral Thesis (1991), Freie Universitat Berlin, biology department). Preferably, the region of the promoter is used that has the nucleotide sequence described in Seq ID No.1.

Apart from the roIC promoter mentioned above the person skilled in the art can without further ado use other promoters for a companion cell specific expression. Further companion cell specific promoters are described in the literature, such as the promoter of the sucrose transporter from Arabidopsis thaliana (Truernit and Sauer, Planta 196 (1995), 564-570.

Furthermore, for different RNAs and proteins their specific occurrence in the companion cells has been described in the literature (see, for example, Foley et al., Plant Mol. Biol. 30 (1996), 687-695; DeWitt, Plant J. 1 (1991), 121-128; Stadler et al., Plant Cell 7 (1995), 1545-1554). Starting from a known protein it is possible for the person skilled in the art without further ado to isolate the cDNA by means of antibodies or by using oligonucleotides derived from the amino acid sequence (cf., e.g., Sambrook et al, Molecular Cloning, A Laboratory Manual, Cold Spring Harbor Laboratory Press (1989), Cold Spring Harbor, N.Y.). Starting from the cDNAs obtained this way it is furthermore possible to screen established genomic libraries from the corresponding organism and to identify genomic fragments. By comparing the nucleotide sequence of the cDNA and of the genomic clone the position of the promoter can be roughly determined. The specificity of the promoter can be verified in a transgenic situation by using chimeric genes consisting of the promoter and indicator genes, such as the β-glucuronidase (cf., e.g., Kertbundit et al., Proc. Natl. Acad. Sci. USA 88 (1991), 5212-5216).

The process according to the invention can in principle be applied to any plant. Therefore, monocotyledonous as well as dicotyledonous plant species are particularly suitable. The process is preferably used with plants that are interesting for agriculture, horticulture and/or forestry.

Examples thereof are vegetable plants such as, for example, cucumber, melon, pumpkin, egg plant, zucchini, tomato, spinach, cabbage species, peas, beans, etc., as well as fruits such as, for example, pears, apples, etc.

Furthermore, oil storing plants are suitable such as, for example, rape, sunflower, soya. In a particularly preferred embodiment starch storing plants are suitable, in particular such as cereals (rice, maize, wheat, rye, oats, triticale, millet, barley), potato, cassava, sweet potato, etc.

The process can also be applied for sucrose storing plants such as, for example, sugar beet and sugar cane, but also for other useful plants such as, for example, cotton, tobacco, types of wood, wine, hops etc.

The invention further relates to recombinant nucleic acid molecules, containing

(a) a region allowing the transcription specifically in the companion cells of plants; and operatively linked thereto

(b) a nucleotide sequence encoding a polypeptide, selected from the group consisting of

(i) sucrose synthases;

(ii) sucrose phosphorylases;

(iii) sucrose transporters;

(iv) proteins the activity of which leads to the stimulation of the proton gradient located at the plasma membrane of plant cells; and

(v) citrate synthases.

With regard to the preferred embodiments of such molecules, the same applies to the region mentioned in (a) and the nucleotide sequence mentioned in (b) what was already mentioned above in connection with the process of the invention.

The invention also relates to vectors containing nucleic acid molecules of the invention, in particular those which are suitable for the transformation of plant cells as well as for the integration of foreign DNA into the plant genome.

The present invention further relates to plant cells transformed with a nucleic acid molecule of the invention and containing it stably integrated into the genome. These cells differ from naturally occurring plant cells for example in that a nucleic acid molecule of the invention is integrated into the genome of the cell at a location where it does not naturally occur.

The invention further relates to transgenic plants containing plant cells of the invention and, due to the expression of the recombinant nucleic acid molecule integrated into the genome in the companion cells of the plants, showing an increased yield in comparison with corresponding non-transformed plants that were cultivated under the same conditions.

The present invention further relates to propagation material of plants of the invention containing the above-described plant cells of the invention. The term “propagation material” in particular comprises seeds, fruits, tubers, rhizomes, cuttings, calli, cell cultures, etc.

Finally, the present invention relates to the use of recombinant nucleic acid molecules containing a region allowing the transcription specifically in the companion cells of plants and, operatively linked thereto, a nucleotide sequence encoding a polypeptide selected from the group consisting of:

(i) proteins with sucrose cleaving enzymatic activity;

(ii) sucrose transporters;

(iii) proteins the activity of which leads to the stimulation of the proton gradient located at the plasma membrane; and

(iv) citrate synthases

for the expression in transgenic plants for increasing the yield. The encoded proteins preferably are the proteins further described above.

Processes for the transformation of monocotyledonous and dicotyledonous plants are known to the person skilled in the art.

For the introduction of DNA into a plant host cell a variety of techniques is available. These techniques comprise the transformation of plant cells with T-DNA using Agrobacterium tumefaciens or Agrobacterium rhizogenes as transformation means, the fusion of protoplasts, the injection, the electroporation of DNA, the introduction of DNA by means of the biolistic method as well as further possibilities.

For the injection and electroporation of DNA in plant cells the plasmids do not have to fulfill specific requirements. Simple plasmids such as pUC derivatives can be used. The use of agrobacteria for the transformation of plant cells has extensively been examined and sufficiently disclosed in the specification of EP-A 120 516, in Hoekema (In: The Binary Plant Vector System Offsetdrukkerij Kanters B. V., Alblasserdam (1985), Chapter V), Fraley et al. (Crit. Rev. Plant. Sci. 4, 1-46) and An et al. (EMBO J. 4 (1985), 277-287).

For the transfer of the DNA to the plant cell plant explants can be co-cultivated with Agrobacterium tumefaciens or Agrobacterium rhizogenes. From the infected plant material (for example leaf explants, segments of stems, roots but also protoplasts or suspension cultivated plant cells) whole plants can be regenerated in a suitable medium which may contain antibiotics or biozides for the selection of transformed cells. The plants obtained that way can then be examined for the presence of the introduced DNA. Other possibilities for the introduction of foreign DNA using the biolistic method or by protoplast transformation are known (cf., e.g., Willmitzer, L., 1993 Transgenic plants. In: Biotechnology, A Multi-Volume Comprehensive Treatise (H. J. Rehm, G. Reed, A. Puhler, P. Stadler, eds.), Vol. 2, 627-659, VCH Weinheim-New York-Basel-Cambridge).

The transformation of dicotyledonous plants via Ti-plasmid-vector systems with the help of Agrobacterium tumefaciens is well-established. Recent studies have indicated that also monocotyledonous plants can be transformed by means of vectors based on Agrobacterium (Chan et al., Plant Mol. Biol. 22 (1993), 491-506; Hiei et al., Plant J. 6 (1994), 271-282; Deng et al., Science in China 33 (1990), 28-34; Wilmink et al., Plant Cell Reports 11 (1992), 76-80; May et al., Bio/Technology 13 (1995), 486-492; Conner and Domisse; Int. J. Plant Sci. 153 (1992), 550-555; Ritchie et al., Transgenic Res. 2 (1993), 252-265).

Alternative systems for the transformation of monocotyledonous plants are the transformation by means of the biolistic method (Wan and Lemaux, Plant Physiol. 104 (1994), 37-48; Vasil et al., Bio/Technology 11 (1993), 1553-1558; Ritala et al., Plant Mol. Biol. 24 (1994), 317-325; Spencer et al., Theor. Appl. Genet. 79 (1990), 625-631), the protoplast transformation, the electroporation of partially permeabilized cells, as well as the introduction of DNA by means of glass fibers.

In particular the transformation of maize is described in the literature several times (cf., e.g., WO95/06128, EP 0 513 849; EP 0 465 875; Fromm et al., Biotechnology 8 (1990), 833-844; Gordon-Kamm et al., Plant Cell 2 (1990), 603-618; Koziel et al., Biotechnology 11 (1993), 194-200). In EP 292 435 and in Shillito et al. (Bio/Technology 7 (1989), 581) a process is described with the help of which and starting from a mucus-free, soft (friable) maize callus fertile plants can be obtained. Prioli and Söndahl (Bio/Technology 7 (1989), 589) describe the regenerating and obtaining of fertile plants from maize protoplasts of the Cateto maize inbred line Cat 100-1.

The successful transformation of other cereal species has also been described, for example for barley (Wan and Lemaux, see above; Ritala et al., see above) and for wheat (Nehra et al., Plant J. 5 (1994), 285-297).

Once the introduced DNA has been integrated into the genome of the plant cell, it usually is stable there and is also contained in the progenies of the originally transformed cell. It usually contains a selection marker which makes the transformed plant cells resistant to a biozide or an antibiotic such as kanamycin, G 418, bleomycin, hygromycin or phosphinotricin and others. Therefore, the individually chosen marker should allow the selection of transformed cells from cells lacking the introduced DNA.

The transformed cells grow within the plant in the usual way (see also McCormick et al., Plant Cell Reports 5 (1986), 81-84). The resulting plants can be cultured normally. Seeds can be obtained from the plants.

Two or more generations should be cultivated to make sure that the phenotypic feature is maintained stably and is transmitted. Seeds should be harvested to make sure that the corresponding phenotype or other properties are maintained.

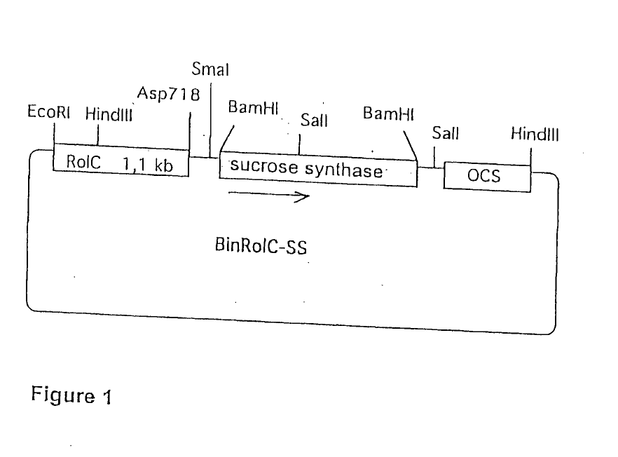

FIG. 1 schematically shows the construction of the plasmid pBinRoIC-SS.

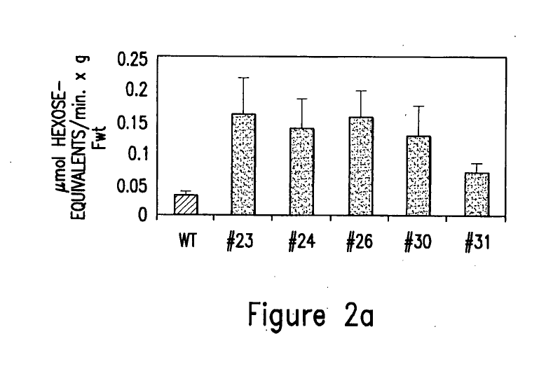

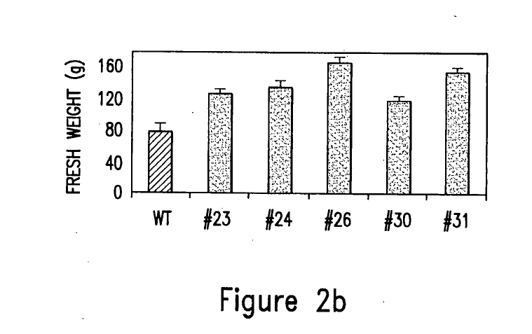

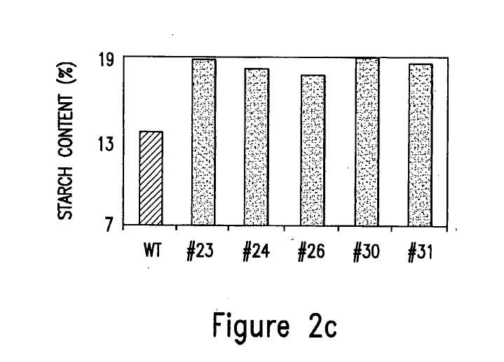

FIG. 2a shows the analysis of the sucrose synthase (SS) activity in leaves of transgenic potato plants which had been transformed with the ROIC-SS construct. The enzyme activity was determined according to Zrenner et al., Plant J. 7 (1995), 97-107). The activity is indicated in μmol hexose equivalents/(min×g fresh weight). The columns represent the average values of three samples per genotype. The standard deviation is also indicated.

FIG. 2b shows the analysis of the tuber yield of transgenic potato plants which had been transformed with the ROIC-SS construct. The columns represent average values of ten to fifteen plants per genotype. The standard deviation is also indicated. The tuber yield is indicated in g per fresh weight.

FIG. 2c shows the analysis of the tuber starch of transgenic potato plants that had been transformed with the ROIC-SS construct. For this purpose tubers harvested from ten to fifteen plants per genotype were collected and the starch content of the tubers was determined according to Von Schéele et al. (Landw. Vers. Sta. 127 (1937), 67-96).

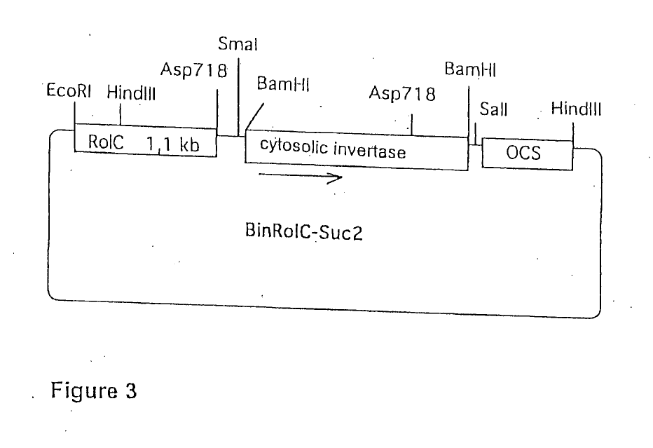

FIG. 3 schematically shows the construction of the plasmid pBinRoIC-Suc2.

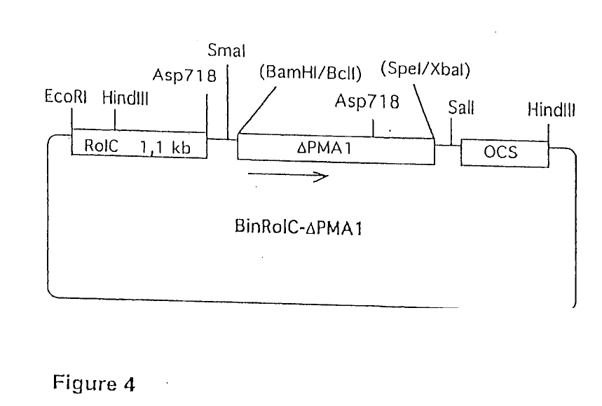

FIG. 4 schematically shows the construction of the plasmid pBinRoIC-ΔPMA1.

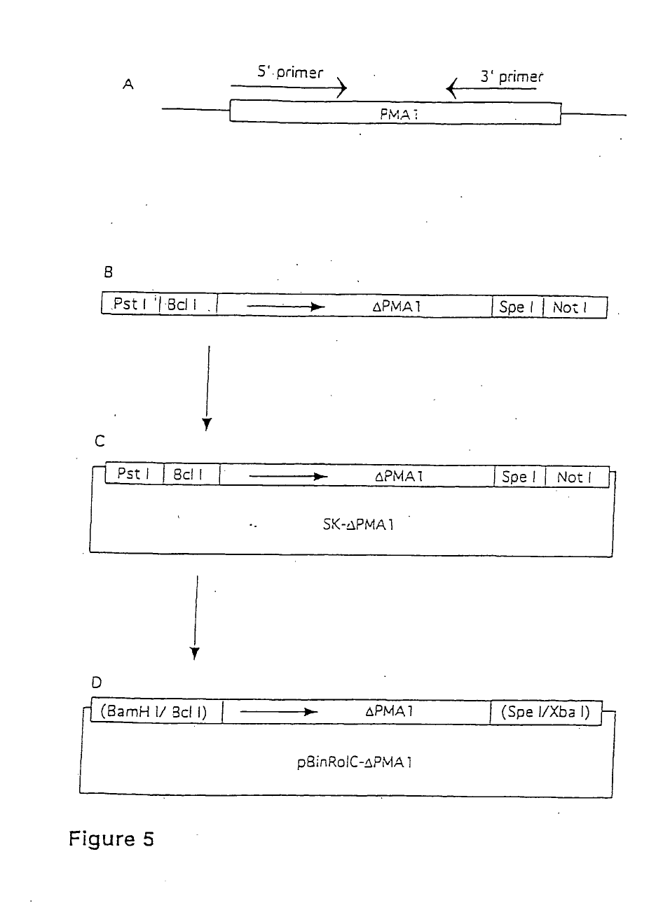

FIG. 5 Schematically shows the cloning strategy of ΔPMA1.

FIG. 5A The H⁺-ATPase ΔPMA1, which was truncated at the 3′ end, was amplified via PCR with the ΔPMA1 cDNA as the matrix and complementary internal primers.

FIG. 5B The flanking cleavage sites of the PCR product were introduced via the correspondingly synthesized primers.

FIG. 5C Pst1/Not1 digestion of the fragment shown in FIG. 5B and cloning of the PCR fragment into the E. coli vector SK- via Pst1/Not1 cleavage sites.

FIG. 5D Bcl1/Spe1 digestion of the plasmid SK-ΔPMA1 as shown in FIG. 5C and cloning of the fragment into the compatible BamHI/XbaI cleavage sites of pBinRoIC.

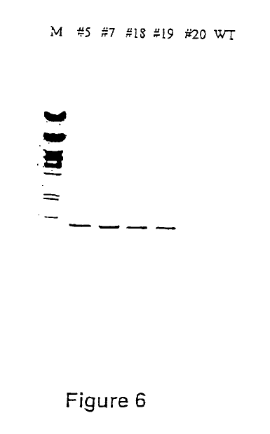

FIG. 6 shows the results of the polymerase chain reaction with specific oligonucleotides indicating the stable integration of ΔPMA1 in the genome of transgenic plants which had been obtained by transformation with the roIC-ΔPMA2 construct. Size of the PCR product=730 bp; WT=wildtype; M=marker.

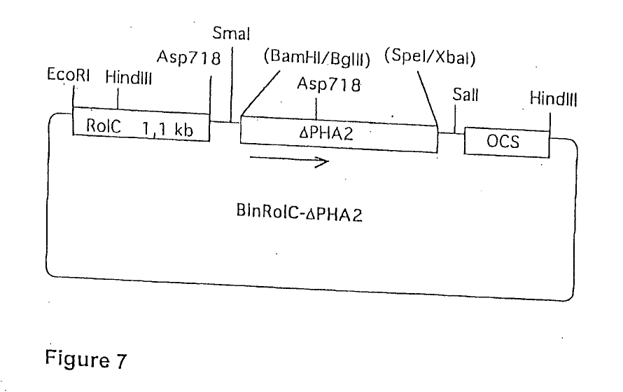

FIG. 7 schematically shows the construction of the plasmid pBinRoIC-ΔPHA2.

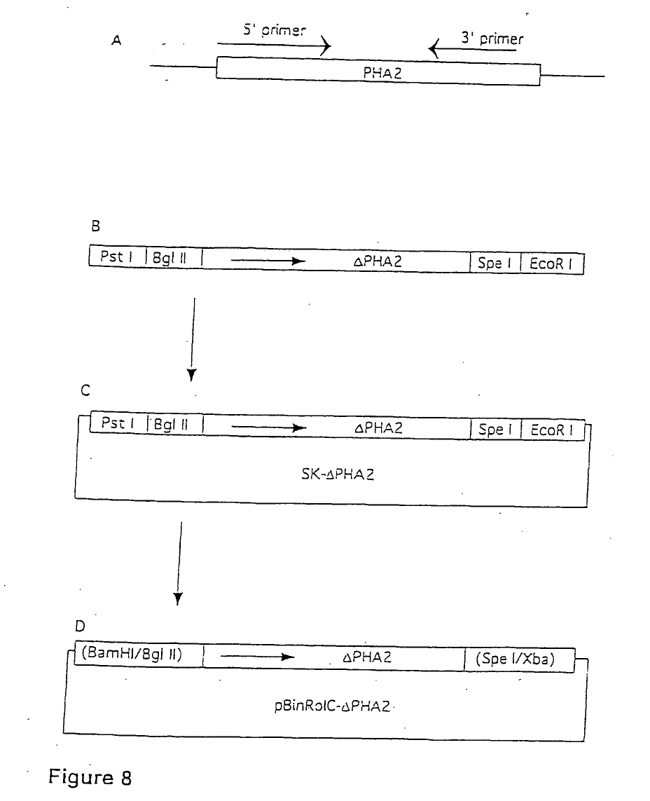

FIG. 8 schematically shows the cloning strategy of ΔPHA2.

FIG. 8A The H⁺-ATPase ΔPHA2, which was truncated at the 3′ end, was amplified via PCR with the PHA2 cDNA as the matrix and complementary internal primers.

FIG. 8B The flanking cleavage sites of the PCR product were introduced via the correspondingly synthesized primers.

FIG. 8C PstI/EcoRI digestion and cloning of the PCR fragment as shown in FIG. 8B into the E. coli vector SK- via PstI/EcoRI cleavage sites.

FIG. 8D BgIII/SpeI digestion of the plasmid SK-ΔPHA2 as shown in FIG. 8C and cloning of the fragment into the compatible BamHI/XbaI cleavage sites of pBinRoIC.

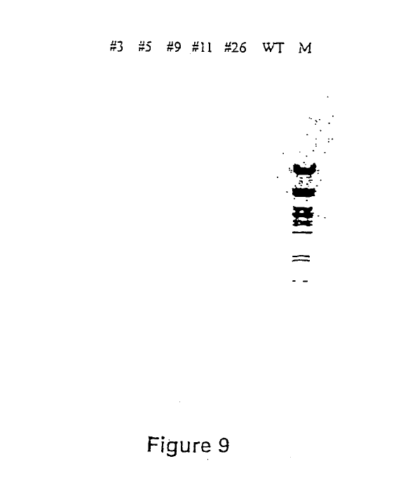

FIG. 9 shows the results of the polyermase chain reaction with specific oligonucleotides indicating the stable integration of ΔPHA2 in the genome of transgenic plants which had been obtained by transformation with the roIC-ΔPHA2 construct. Size of the PCR product=758 bp; WT=wildtype; M=marker.

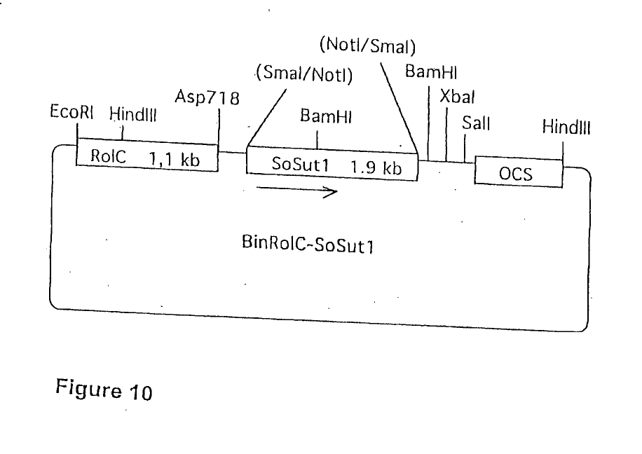

FIG. 10 schematically shows the construction of the plasmid pBinRoIC-SoSUT1.

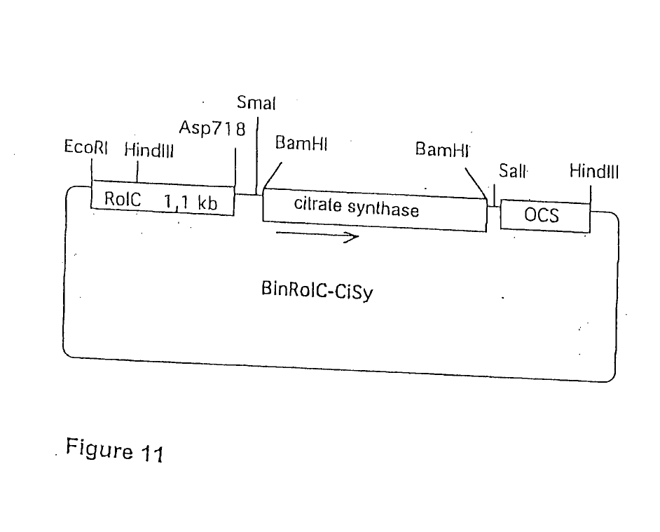

FIG. 11 schematically shows the construction of the plasmid pBinRoIC-CiSy.

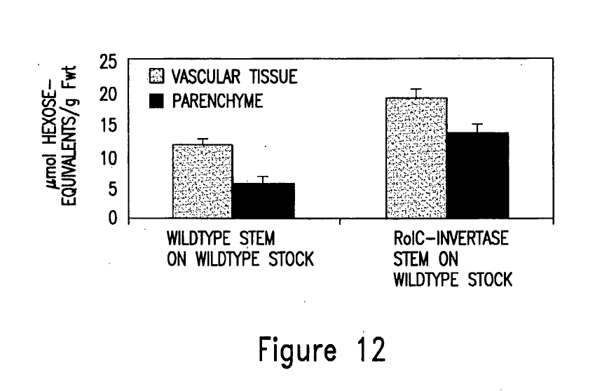

FIG. 12 shows the results of the determination of the sucrose content in parenchymatic samples of tubers of engrafted potato plants enriched with vascular tissue. The genotypes used for engrafting are the lines RoIC-Suc2-#25 (cytosolic invertase) and wildtype Solanum tuberosum var. Désirée. The sucrose content was determined according to Stitt et al. (Methods Enzymol. 174 (1989), 518-522). The columns represent the average values of 12 samples per engrafted type. The standard deviation is indicated. The sucrose content is indicated as μmol hexose equivalents/g fresh weight.

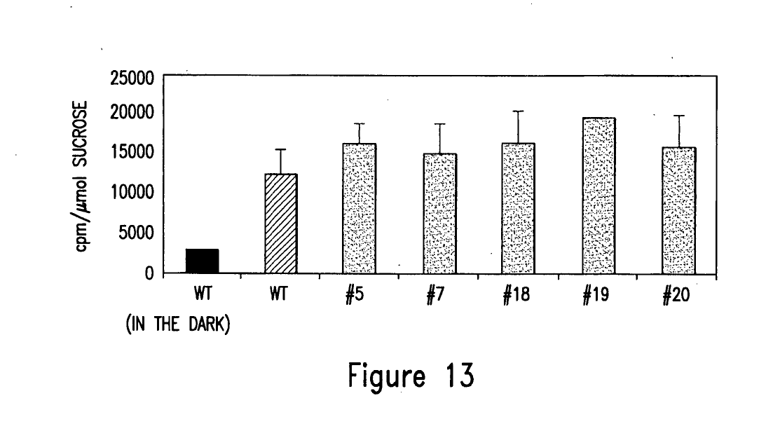

FIG. 13 shows the analysis of phloem exudates of ΔPMA1 leaves which were kept under light for six hours in a 14CO₂ atmosphere. The sucrose content was determined according to Stitt et al. (loc. cit). The columns represent the average values of four to five samples per genotype. The standard deviation is indicated.

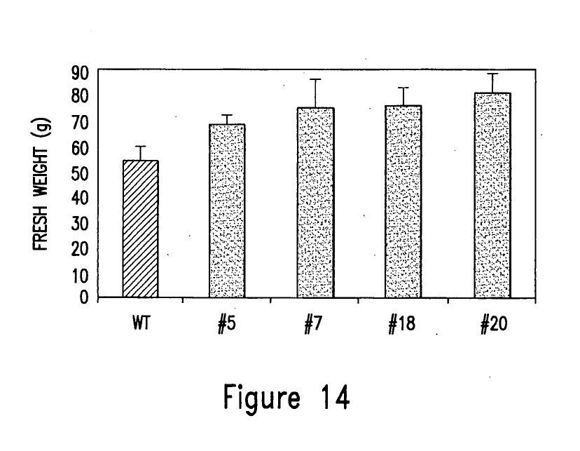

FIG. 14 shows the tuber yield (in gram fresh weight) of ΔPMA1 plants. The columns represent the average values of six plants per genotype. The standard deviation is indicated. The tuber yield is indicated in g fresh weight.

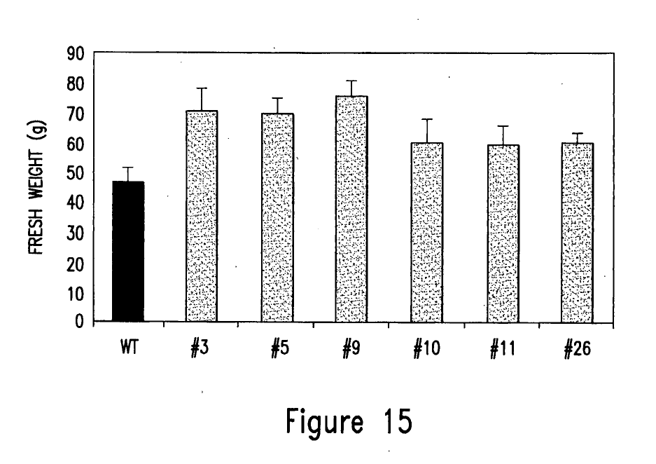

FIG. 15 shows the tuber yield (in gram fresh weight) of ΔPHA2 plants. The columns represent average values of four to five plants per genotype. The standard deviation is indicated. The tuber yield is indicated in g fresh weight.

The following examples illustrate the invention.

### EXAMPLE 1

### Production of the Plasmid pBinRoIC-SS and Production of Transgenic Potato Plants

The plasmid pBinRoIC-SS contains the three fragments A, B and C in the binary vector pBin19 (Bevan, Nucl. Acids Res. 12 (1984), 8711) (cf. FIG. 1).

The fragment A comprises the roIC promoter from Agrobacterium rhizogenes. The roIC promoter contains as an EcoRI/Asp718 DNA fragment of 1138 bp (Lerchl et al., Plant Cell 7 (1995), 259-270) the DNA region (position: 11306 to position 12432) of the TL-DNA of the Ri-agropin-type plasmid from A. rhizogenes (Slightom et al., J. Biol. Chem. 261 (1986), 108-121). The fragment A is inserted into the EcoRI and Asp718 cleavage sites of the polylinker of pBin19.

The fragment B contains the coding region (position: 76 to position 2493) of the cDNA of the sucrose synthase (SS) from Solanum tuberosum (Salanoubat and Belliard, Gene 60 (1987), 47-56). The fragment B was obtained as BamHI fragment of 2427 bp from the vector pBluescript SK⁻, in which it is inserted into the BamHI cleavage site of the polylinker. The fragment B was inserted in sense orientation in the vector pBin19 into the BamHI cleavage site, that is downstream of the roIC promoter in an orientation allowing the transcription of a translatable RNA.

The fragment C contains the polyadenylation signal of the Gene 3 of the T-DNA of the Ti plasmid pTi ACH 5 (Gielen et al., EMBO J. 3 (1984), 835-846), in particular the nucleotides 11749-11939, which was isolated as a PvuII/HindIII fragment from the plasmid pAGV 40 (Herrera-Estrella et al., Nature 303 (1983), 209-213) and which upon addition of SphI linkers was cloned into the PvuII cleavage site between the SphI and the HindIII cleavage site of the polylinker of pBin19.

The resulting plasmid pBinRoIC-SS was introduced into potato plant cells via the gene transfer mediated by Agrobacterium tumefaciens. For this purpose ten small leaves of a potato sterile culture (Solanum tuberosum L. cv.) Désirée) wounded with the scalpel were put into 10 ml MS medium (Murashige and Skoog, Physiol. Plant. 15 (1962), 473 with 2% of sucrose containing 50 μl of a Agrobacterium tumefaciens overnight culture grown under selection. After 3 to 5 minutes of gentle shaking a further incubation followed for two days in the dark. Then the leaves were put on MS medium with 1.6% glucose, 5 mg/l naphtyl acetic acid, 0.2 mg/l benzylaminopurin, 250 mg/l claforan, 50 mg/l kanamycin and 0.8% bacto-agar for callus induction. After an incubation of one week at 25° C. and 3000 lux the leaves were put on MS medium with 1.6% glucose, 1.4 mg/l zeatin ribose, 20 μg/l naphtyl acetic acid, 20 μg/l giberellic acid, 250 mg/l claforan, 50 mg/l kanamycin and 0.8% bacto-agar.

The analysis of the leaves of a number of plants transformed with this vector system unambiguously indicated the presence of an increased sucrose synthase activity. This is a result of the expression of the sucrose synthase gene from potato contained in pBinRoIC-SS (cf. FIG. 2a).

The analysis of the tuber yield (tuber fresh weight in gram) of plants transformed with this vector system and showing an increased sucrose synthase activity unambiguously showed an increased tuber yield. This is also a result of the expression of the sucrose synthase gene from potato contained in pBinRoIC-SS (cf. FIG. 2b).

The starch content of potato tubers is linearly dependent on the density of the tubers (von Schéele et al., Landw. Vers. Sta. 127 (1937), 67-96). The analysis of the density of transgenic tubers of plants which had been transformed with the vector system pBinRoIC-SS having an increased sucrose synthase activity surprisingly showed an increased starch content. This is a result of the expression of the sucrose synthase gene from potato contained in pBinRoIC-SS (cf. FIG. 2c).

### EXAMPLE 2

### Production of the Plasmid pBinRoIC-Suc2 and Production of Transgenic Potato Plants

The plasmid pBinRoIC-Suc2 contains the three fragments A, B and C in the binary vector pBin19 (Bevan, loc. cit.) and is illustrated in FIG. 3.

The fragments A and C correspond to the fragments A and C as described in Example 1.

The fragment B contains the coding region (position: 845 to position: 2384) of the gene of the cytosolic invertase from yeast (Saccharomyces cerevisiae). The fragment B was obtained as a BamHI fragment with a length of 1548 bp from the vector pBluescript SK⁻ in which it is inserted in the BamHI cleavage site of the polylinker. The fragment B is inserted in sense orientation into pBin19 in the BamHI cleavage site.

The plasmid pBinRoIC-Suc2 was introduced into potato plant cells via gene transfer mediated by Agrobacterium. From transformed cells whole plants were regenerated. Such plants show in comparison to non-transformed plants an increased yield (increased biomass).

### EXAMPLE 3

### Production of the Plasmid pBinRoIC-ΔPMA1 and Production of Transgenic Potato Plants

The plasmid pBinRoIC-ΔPMA1 contains the three fragments A, B and C in the binary vector pBin19 (Bevan, loc. cit.) and is schematically illustrated in FIG. 4.

The fragments A and C correspond to the fragments A and C as described in Example 1. The fragment B contains the coding region (position: 937 to position: 3666) of the gene of the proton ATPase PMA1 from the yeast Saccharomyces cerevisiae (Serrano et al., Nature 319 (1986), 689-693). The fragment B was obtained by means of polymerase chain reaction (PCR). For this purpopse the 3′ end of the coding region of the gene PMA1 was truncated on purpose by 27 bp and at the same time a necessary new stop codon was introduced. The DNA fragment modified this way was called ΔPMA1. The fragment B was inserted, as a BclI/SpeI fragment with a length of 2739 bp, in sense orientation into the BamHI (compatible insertion site for BclI restriction sites) and XbaI (compatible insertion site for SpeI restriction sites) cleavage sites of the vector pBin19.

The fragment B was obtained as BclI/SpeI fragment from the vector pBluescript SK⁻ in which it is inserted via the cleavage sites NotI and PstI of the polylinker (cf. FIG. 5). The plasmid pBinRoIC-ΔPMA1 was introduced into potato plant cells via the gene transfer mediated by Agrobacterium. Whole plants were regenerated from transformed cells.

The stable integration of ΔPMA1 in the genome of transgenic plants which had been obtained by using the vector system pBinRoIC-ΔPMA1 was detected by means of polymerase chain reaction (PCR) (cf. FIG. 6).

The transformed plants show an increased yield (increased biomass) in comparison to non-transformed plants (see FIGS. 13 and 14).

### EXAMPLE 4

### Production of the Plasmid pBinRoIC-ΔPHA2 and Production of Transgenic Potato Plants

The plasmid pBinRoIC-ΔPHA2 contains the three fragments A, B and C in the binary vector pBin19 (Bevan, loc. cit.) and is schematically illustrated in FIG. 7.

The fragments A and C correspond to the fragments A and C as described in Example 1.

The fragment B contains the coding region (position: 64 to position: 2672) of the cDNA of the proton-ATPase PHA2 (Harms et al., Plant Mol. Biol. 26 (1994), 979-988). The fragment B was obtained by means of polymerase chain reaction (PCR). For this purpose the 3′ end of the coding region of the gene PHA2 was on purpose truncated by 249 bp, and at the same time two new stop codons were introduced. The DNA fragment modified that way was called ΔPHA2. The fragment B was inserted in sense orientation as a BgIII/SpeI fragment with a length of 2631 bp into the BamHI (compatible insertion site for BgIII restriction sites) and XbaI (compatible insertion site for SpeI restriction sites) cleavage sites of the vector pBin19.

The fragment B was obtained as BgIII/SpeI fragment from the vector pBluescript SK⁻, in which it is inserted into the EcoRI and PstI cleavage sites of the polylinker sequence (cf. FIG. 8: cloning strategy ΔPHA2).

The plasmid pBinRoIC-ΔPHA2 was introduced into potato plant cells via the gene transfer mediated by Agrobacterium. Whole plants were regenerated from transformed cells.

The stable integration of ΔPHA2 in the genome of transgenic plants which had been obtained using the vector system pBinRoIC-ΔPHA2 was detected by means of polymerase chain reaction (PCR) (cf. FIG. 9).

The transformed plants show an increased yield (increased biomass) in comparison to non-transformed plants (see FIG. 15).

### EXAMPLE 5

### Production of the Plasmid pBinRoIC-SoSUT1 and Production of Transgenic Potato Plants

The plasmid pBinRoIC-SoSUT1 contains the three fragments A, B and C in the binary vector pBin19 (Bevan, loc. cit.) and is schematically illustrated in FIG. 10.

The fragments A and C correspond to the fragments A and C as described in Example 1.

The fragment B contains the cDNA (position: 1 to position: 1969) encoding a sucrose transporter from spinach (Spinacia oleracea) (Riesmeier et al., EMBO J. 11 (1992), 4705-4713; accession number X67125 and S51273). The fragment B was obtained as a NotI fragment from the vector pBluescript SK⁻, in which it is inserted via a NotI linker sequence. For the cloning into the SmaI cleavage site of the vector pBin19 the sticky ends of the fragment resulting from the NotI digestion were converted to blunt ends and inserted in sense orientation into the pBin19. The resulting plasmid was called pBinRoIC-SoSUT1.

It was introduced into potato plant cells via the gene transfer mediated by Agrobacterium. Whole plants were regenerated from transformed cells. Plants transformed that way show an increased yield (increased biomass) in comparison to non-transformed plants.

### EXAMPLE 6

### Production of the Plasmid pBinRoIC-CiSy and Production of Transgenic Potato Plants

The plasmid pBinRoIC-CiSy contains the three fragments A, B and C in the binary vector pBin19 (Bevan, Nucl. Acids Res. 12 (1984), 8711) modified according to Becker, Nucl. Acids Res. 18 (1990), 203 (cf. FIG. 11).

The fragment A comprises the roIC promoter from Agrobacterium rhizogenes. The roIC promoter contains as an EcoRI/Asp718 DNA fragment with a length of 1143 bp (Lerchl et al., The Plant Cell 7 (1995), 259-270) the DNA region (position: 11306 to position 12432) of the TL-DNA of the Ri-agropin type plasmid from A. rhizogenes (Slightom et al., J. Biol. Chem. 261 (1986), 108-121). The fragment A is inserted in the EcoRI and Asp718 cleavage sites of the polylinker of pBin19.

The fragment B contains the coding region of the cDNA of the citrate synthase (CiSy) from the fission yeast Saccharomyces cerevisiae. The fragment B was obtained as a BamHI fragment with a length of 1400 bp from the vector pBluescript SK-, in which it is inserted in the BamHI cleavage site of the polylinker (Landschütze, Studies on the influence of the acetyl-CoA synthesis and use in transgenic plants, Doctoral Thesis, Freie Universität Berlin, (1985) D83/FB15 No. 028).

The fragment C contains the polyadenylation signal of the gene 3 of the T-DNA of the Ti plasmid pTiACH 5 (Gielen et al., EMBO J. 3 (1984), 835-846), nucleotides 11749-11939, which had been isolated as a PvuII/HindIII fragment from the plasmid pAGV 40 (Herrera-Estrella et al., Nature 303 (1983), 209-213) and which after addition of SphI linkers to the PvuII cleavage site had been cloned between the SphI and HindIII cleavage site of the polylinker of pBin19.

The plasmid pBinRoIC-CiSy has a length of about 13 kb.

The plasmid pBinRoIC-CiSy was inserted into potato plants via the gene transfer mediated by Agrobacterium tumefaciens. Whole plants were regenerated from transformed cells.

The analysis of a number of plants transformed with this vector system unambiguously showed an increased biomass, which is a result of the expression of the CiSy cDNA from yeast contained in pBinRoIC-CiSy.

### EXAMPLE 7

### Grafting Experiment

For grafting the shoot of a receiver plant is replaced with the shoot of a donor plant.

In this experiment the shoot of a transgenic plant (RoIC-Suc2 #25) is grafted onto the base of a wildtype plant (Solanum tuberosum, var. Désirée). In a control experiment a wildtype shoot is grafted onto a wildtype base in order to rule out culturing differences in the experiments (autografting). The aim of the experiment is to examine the exclusive impact of the photosynthetic activity and photoassimilate distribution of a transgenic shoot on organs (in this case tubers) of a wildtype base. Potato plants were transferred from a tissue culture to earth and placed into a greenhouse. After approx. five weeks (the plants have not yet induced tuber production at this stage) the plants are grafted. For this purpose the shoot of the receiver plant which is not needed is cut off, and a wedge is cut into the stem of the receiver plant. The donor shoot to be grafted is cut at the stem end in the appropriate way and is inserted into the wedge of the receiver plant. The grafting site is fixed with an adhesive tape.

Then the grafted potato plants are kept under increased air humidity and in shadow for approx. one week. Within seven to ten days they are step by step adapted to normal greenhouse conditions. At this stage the plants are seven weeks old. All leaves of the receiver plant are now removed, and the stem is covered from light with an aluminum sheet in order to guarantee that exclusively the photosynthetic activity and the photoassimilate distribution of the donor shoot nourishes the base of the grafted plant.

The plants are kept in the greenhouse until the potato tubers are harvested approx. two months after the grafting and approx. three months after the planting into soil. The results of such a grafting experiment are illustrated in FIG. 12.

1. A process for increasing the yield of plants comprising transforming a plant with at least one recombinant DNA construct comprising
(a) a region allowing the transcription specifically in the companion cells; and operatively linked thereto
(b) a nucleotide sequence encoding a polypeptide selected from the group consisting of:
(i) proteins with an enzymatic activity that cleaves sucrose;
(ii) sucrose transporters;
(iii) proteins the activity of which leads to the stimulation of the proton gradients located at the plasma membrane of plant cells; and
(iv) citrate synthases;
wherein said at least one construct is stably integrated into the genome of said plant and wherein the expression of said nucleotide results in an increase in plant yield.

2. The process of claim 1, wherein the nucleotide sequence encodes a plant protein.

3. The process of claim 1, wherein the nucleotide sequence encodes a protein from a bacterium or a fungus.

4. The process of claim 1, wherein the nucleotide sequence encodes a protein with an enzymatic activity that cleaves sucrose, selected from the group consisting of sucrose synthases, sucrose phosphorylases and invertases.

5. The process according to claim 1, wherein the region mentioned in (a) is the roIC promoter from Agrobacterium rhizogenes.

6. A recombinant nucleic acid molecule comprising
(a) a region allowing the transcription specifically in the companion cells of plants; and operatively linked thereto
(b) a nucleotide sequence encoding a polypeptide selected from the group consisting of
(i) sucrose synthases;
(ii) sucrose phosphorylases;
(iii) sucrose transporters;
(iv) proteins the activity of which leads to the stimulation of the proton gradient located at the plasma membrane of plant cells; and
(v) citrate synthases,
wherein said recombinant nucleic acid molecule, when stably integrated into the genome of plants and expressed, leads to an increase of the yield of plants.

7. A vector comprising the recombinant nucleic acid molecule of claim 6.

8. The vector of claim 7, wherein the vector is suitable for transformation of plant cells and for integration of foreign DNA into the plant genome.

9. A plant cell transformed with and comprising a recombinant nucleic acid molecule of claim 6.

10. A plant comprising plant cells of claim 9, wherein the plant shows an increased yield in comparison to a corresponding non-transformed plant due to the expression of the recombinant nucleic acid molecule in the companion cells of the plant.

11. Propagation material of a plant of claim 10.

12. Use of a recombinant nucleic acid molecule containing a region allowing the transcription specifically in the companion cells of plants and operatively linked thereto a nucleotide sequence encoding a polypeptide selected from the group consisting of
(i) proteins with an enzymatic activity that cleaves sucrose;
(ii) sucrose transporters;
(iii) proteins the activity of which leads to the stimulation of the proton gradient located at the plasma membrane; and
(iv) citrate synthases, for the expression in transgenic plants for increasing the yield.
